(19) United States
(12) Patent Application Publication
(10) Pub. No.: US 2003/0134303 A1
(43) Pub. Date: Jul. 17, 2003

(54) Adenine nucleotide-binding protein-directed probes, and methods of their synthesis and use

(75) Inventors: David A. Campbell, San Diego, CA (US); Paul Wash, San Diego, CA (US)

Correspondence Address: FOLEY & LARDNER, P.O. BOX 80278, SAN DIEGO, CA, 92138-0278, US

(73) Assignee: ActivX Biosciences, Inc,

(21) Appl. No.: 10/213,359

(22) Filed: Aug. 5, 2002

Related U.S. Application Data
(60) Provisional application No. 60/339,424, filed on Dec. 11, 2001.

(51) Int. Cl. C12Q 1/68; G01N 33/53

(52) U.S. Cl. 435/6; 435/7.1

(57) ABSTRACT

The invention relates to compositions and methods for the synthesis and use of Adenosine nucleotide binding protein-directed affinity labels. Adenosine nucleotide binding proteins may be labeled with probes comprising adenosine, or an analogue thereof, functionalized at the 5′ position with reactive group capable of reacting with an amino acid side chain functionality at an adenosine nucleotide binding site, and at the 2′ or 3′ position with a TAG for sequestering and/or identifying the resulting conjugate. The probes may be used for determining the presence or amount of one or more adenosine nucleotide binding proteins in a complex mixture, particularly a cellular mixture, for screening for drugs, and other purposes associated with the presence of the adenine nucleotide-binding protein(s) in a cell or changes in the presence, amount, activity, or relative concentration of the active adenosine nucleotide-binding protein.

### FIELD OF THE INVENTION

The invention relates generally to compositions and methods for labeling adenine nucleotide-binding proteins, preferably ATP binding proteins, and most preferably kinases.

### BACKGROUND INFORMATION

Proteomics, and the desire to understand the protein profile of cells, tissues, organs, and/or organisms, has become a major interest in research and the field of medicine. The biology of cells during their life cycle, cellular proteomic response to changes in maturation and differentiation, function and location, changes in environment, such as the presence of drugs, microorganisms, etc., and diseased states are all of importance in maintaining health. Now that the human genome is substantially determined, proteomics is the next stage in analyzing how the genome operates. There are many different proteins associated with synthesizing compounds, modifying proteins, degrading proteins, initiating transcription, folding and modifying nascent proteins, translocating cellular components, transport into and out of the cell, and sensing cellular status, among a host of other functions.

In studying proteomics, there are various levels of sophistication. One may study the rate of transcription of the messenger level for a particular protein as a surrogate for the amount of protein in the cell. There are numerous methods for identifying and quantifying particular ribonucleic acid sequences in the cell and by observing variations in the amount of mRNA present, one may assume that the amount of the protein is changing in the same direction. However, mRNAs may be spliced differently, so that one must know the sequence for each of the differently spliced mRNAs to be sure that one is following the correct protein. In addition, mRNA levels may not correlate with protein levels in cells.

Alternatively, there are numerous assays for determining the total amount of protein, particularly using antibodies that recognize an epitope of the protein. This level of sophistication provides more information than following the mRNA, but ignores the many modifications that a protein may undergo to be active, as well as denatured protein and protein that has been designated for degradation, e.g. ubiquitinated protein. Phosphorylation, dephosphorylation, acetylation, prenylation, farnesylation, reduction and oxidation are all processes that may be necessary for the protein to be in its active form.

Frequently, to be of significant value, one needs to know the amount of protein that is in an active conformation or has been modified to provide the active protein. In other situations, one wishes to know whether an active site is in the proper conformation and available for reaction, particularly an enzyme with a substrate or cofactor. In some circumstances one wishes to determine the presence and status of a group of proteins, particularly members of a family, rather than having to do individual assays with individual probes for each target.

Among protein groups are those that bind adenine nucleotides (e.g., ATP, ADP, and AMP). This group includes enzymes that bind ATP and catalyze the transfer of a phosphate group from ATP to another substrate molecule, particularly the protein kinases that autophosphorylate or phosphorylate other proteins. Adenine nucleotide-binding proteins play an essential role in the biology of the cell, since phosphorylation is a major aspect of regulation of the cells metabolism and response to external stimuli. In addition, adenine nucleotide-binding proteins are essential for providing energy to endothermic steps in the activities of the cells. There is, therefore, substantial interest in developing methods that permit the accurate determination of adenine nucleotide-binding protein profiles in cells in a rapid and quantitative manner.

The importance of adenine nucleotide-binding proteins has stimulated the development of numerous affinity labels for, e.g., kinases and other ATP binding enzymes, using adenine and various modifications of the remainder of ATP. The following are representative publications and the labels disclosed: p-fluorosulfonylbenzoyl adenosine (Mueller, et al., Biochemistry 1999, 38, 9831-9); α- or γ-³²P X(Y)₄-8-N₃-ATP, where X is Co or Cr and Y is NH₃ or H₂O respectively (Antolovic, et al., J. Biochem. 1999, 261, 181-9); 2′(3′)-O-(2,4,6-trinitrophenyl) ADP (Martin, et al., J. Biol. Chem. 2000, 275, 24512-7); adenosine diphosphopyridoxal (Kaguta, et al., FEBS Lett. 1998, 427, 377-80); 2′,3′-dialdehyde ATP (Li, et al., Biochimie 1997, 79, 221-7); 5-(p-fluorosulfonylbenzoyl) adenosine-2′(3′)-methylanthraniloyl (Scoggins, et al., Biochemistry 1996, 35, 9197-203); 2-[3H]-8-azido ATP (Schrattenholz, et al., J. Recept. Res. 1994, 14, 197-208); herbimycin A (Fukazawa, et al., FEBS Lett. 1994, 340, 155-8); 2-azidoadenosine-5-triphosphate and 3′-O-(5-fluoro-2.4-dinitrophenyl) ATP (Mayinger, et al., Biochemistry 1992, 31, 10536-43); γ-[4-(N-2-chloroethyl-N-methylamino)benzylamide ATP ¹⁴C (James, et al., FEBS Lett. 1990, 273, 139-43) and Vollmer and Colman, Biochemistry 1990, 29, 2495-501).

### SUMMARY OF THE INVENTION

The present invention provides compositions and methods for assessing profiles of adenine nucleotide-binding proteins in biological samples. In various embodiments, one or more samples, most preferably one or more complex protein mixtures as defined below, are contacted with one or more probes directed to the adenine nucleotide binding portion of one or more adenine nucleotide-binding proteins. The binding selectivity of the probe(s) may be selected to allow the skilled artisan to analyze the presence, amount, and/or activity of a selected portion of the adenine nucleotide-binding proteins present in a sample, thereby simplifying the analysis of complex protein mixtures.

The methods and compositions described herein relate to probes, referred to herein as “adenine nucleotide-binding protein-directed affinity probes” or “ANBPs” having an affinity moiety for directing the binding of the ANBPs to one or more adenine nucleotide-binding proteins, preferably protein kinases, a reactive group for forming a covalent bond at or near the nucleotide binding site, and a TAG (e.g., a detectable label, preferably a fluorophore).

One or more ANBPs are combined with a protein-containing sample under conditions for binding and reaction of the ANBP(s) with target adenine nucleotide-binding proteins that are present in the sample. The resulting products are then used to assess the adenine nucleotide-binding protein profile of the sample, and can be correlated to the presence, amount, or activity of one or more target adenine nucleotide-binding proteins present in the original complex protein mixture.

In a first aspect, the present invention relates to methods and compositions for determining a profile (e.g., a determination of the presence, amount, activity, and/or relative abundance) of one or more adenine nucleotide-binding proteins in a complex protein mixture. These methods comprise contacting the complex protein mixture with one or more distinct ANBPs, each of which corresponds to one or more target adenine nucleotide-binding proteins. Each ANBP preferably comprises an affinity moiety that is adenosine, or an analogue thereof, conjugated to an identifiable TAG, such as a ligand or a detectable label, and a reactive group that reacts with a target adenosine nucleotide-binding protein when the ANBP binds to that target protein. The adenosine nucleotide-binding protein profile can then be determined by, for example, sequestering probe-target protein conjugates through binding the TAG moiety to a conjugate receptor, or by detecting a signal from the detectable TAG in the probe-target protein conjugates.

In preferred embodiments, the reactive group of an ANBP is attached via a linking group through the 5′ position of the adenosine or adenosine analogue. Particularly preferred reactive groups include fluorosulfonyl, vinylsulfonyl, acryl, and chloroacetyl groups. In various embodiments, the identifiable TAG may be linked to the 2′ or 3′ position of the adenosine or adenosine analogue, preferably through a linking group. Exemplary identifiable TAGs are described hereinafter.

In another aspect, the present invention relates to ANBP compounds comprising adenosine or an adenosine analogue conjugated to an identifiable TAG, such as a ligand or a detectable label, and a reactive group that reacts with one or more target adenine nucleotide-binding proteins when the ANBP(s) bind(s) to a target adenosine nucleotide-binding protein. Exemplary compositions are described hereinafter.

In yet another aspect, the present invention relates to compositions comprising one or more ANBPs bound to one or more target adenosine nucleotide-binding proteins in a complex protein mixture.

In preferred embodiments, the ANBP-adenine nucleotide-binding protein conjugates can be separated from other components of the complex protein mixture, for example by sequestering one or more conjugates (e.g., by binding to a receptor that binds the TAG portion of the ANBP or by using a “tethered” ANBP), by chromatographic methods, by mass spectrographic methods, and/or by other means such as electrophoresis.

In yet other embodiments, following reaction of the complex protein mixture with one or more ANBPs, the resulting ANBP-adenine nucleotide-binding protein conjugates may be proteolytically digested to provide ANBP-labeled peptides. This digestion may occur while the protein conjugates are sequestered to a solid phase, or while free in solution. In preferred embodiments, ANBP s are selected such that each target adenine nucleotide-binding protein forms a conjugate with a single ANBP, most preferably at a single discrete location in the target nucleotide binding protein; thus, each conjugate gives rise to a single ANBP-labeled peptide. Enrichment, separation, or identification of one or more ANBP-labeled peptides may be achieved using liquid chromatography and/or electrophoresis. Additionally, mass spectrometry may be employed to identify one or more ANBP-labeled peptides by molecular weight and/or amino acid sequence. In particularly preferred embodiments, the sequence information derived from the ANBP-labeled peptide(s) is used to identify the adenine nucleotide-binding protein from which the peptide originally derived. Variations of these aspects can involve the comparison of two or more proteomes, e.g., with ANBPs having different TAGs, or, when analysis comprises mass spectrometry, having different isotopic compositions.

In yet another aspect, the present invention relates to methods for comparing the presence, amount, or activity of one or more target adenine nucleotide-binding proteins in two or more complex protein mixtures using the methods and compositions described herein. In various embodiments, these methods comprise one or more of the following steps: contacting one or more complex protein mixture(s) with one or more ANBPs, where the ANBP(s) specifically bind to one or more target adenine nucleotide-binding proteins present in each complex protein mixture; combining the complex protein mixtures following this contacting step to form a combined complex protein mixture; prior to and/or following this combination, removing one or more non-sequestered components of the complex protein mixture(s). The adenine nucleotide-binding protein profile can then be determined by analyzed by the screening and/or identification methods described hereinafter.

In preferred embodiments, the methods and compositions described herein are applied to determining the adenine nucleotide-binding protein profiles of cancerous and other diseased tissue by obtaining one or more samples of diseased tissue, and determining the adenine nucleotide-binding protein profile of the tissue sample(s). In particularly preferred embodiments, the adenine nucleotide-binding protein profile of diseased tissues can be compared to that of normal tissue sample(s) to determine differences in the profiles of the two tissue samples.

In still another aspect, the present invention relates to methods and compositions for detecting disease in a test sample. In preferred embodiments the test sample will be a cell or tissue sample. In particularly preferred embodiments, the tissue sample will be a neoplasmic sample and the disease is a cancer. The methods involve determining the target adenine nucleotide-binding protein profile of the test sample; comparing the profile of the test sample with the corresponding profile of a known non-diseased sample and/or with the profile of a known diseased sample; and determining whether the test sample is in a state of disease. A “non-diseased” sample is a sample of cells or tissues that is known to not have the disease being tested for. It is preferably a normal, healthy sample of the cells or tissue.

In another aspect the present invention provides methods of determining the inhibitory and/or stimulatory potency of a test compound against one or more target adenine nucleotide-binding protein. The methods involve contacting one or more ANBPs with a test sample containing the test compound and the target adenine nucleotide-binding protein(s); allowing the ANBPs to react with proteins contained in the test sample; and detecting a signal that indicates the level of the target adenine nucleotide-binding protein(s) available for labeling in the test sample.

In preferred embodiments, this protein level is compared to the level of the target adenine nucleotide-binding protein(s) available for labeling in the absence of the test compound. By such methods, the inhibitory and/or stimulatory potency of the test compound against the target adenine nucleotide-binding protein(s) can be determined. The “inhibitory potency” is the extent to which the presence of the compound causes the inhibition of target nucleotide binding protein activity, while “stimulatory potency” is the extent to which the presence of the compound causes an increase in target nucleotide binding protein activity.

In yet another aspect, the present invention provides kits for performing the methods described. The kits contain one or more of the materials described for conducting the methods. The kits can include ANBPs in the solid phase or in a liquid phase (such as buffers provided) in a package. The kits also can include buffers for preparing solutions for conducting the methods, and pipettes for transferring liquids from one container to another. By “package” is meant material enveloping a vessel containing the ANBPs. In preferred embodiments, the package can be a box or wrapping. The kit can also contain items that are not contained within the package but are attached to the outside of the package, for example, pipettes.

The summary of the invention described above is not limiting and other features and advantages of the invention will be apparent from the following detailed description of the preferred embodiments, as well as from the claims.

### BRIEF DESCRIPTION OF THE FIGURES

FIG. 1 describes schematically the synthesis of 5′-amido ATP-based ANBPs.

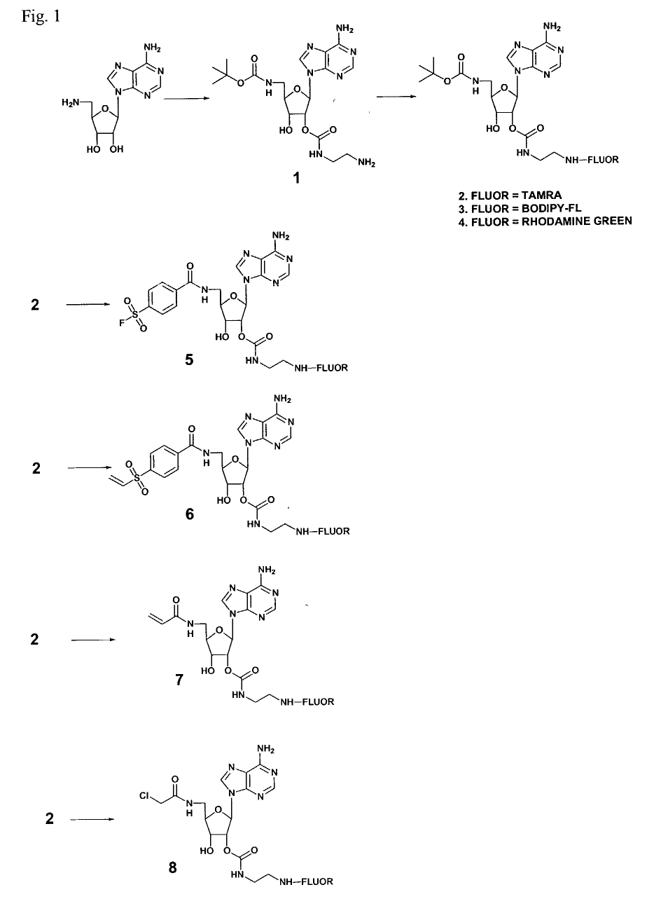

FIG. 2. describes schematically the synthesis of 5′-amido and 5′-ester ATP-based ANBPs.

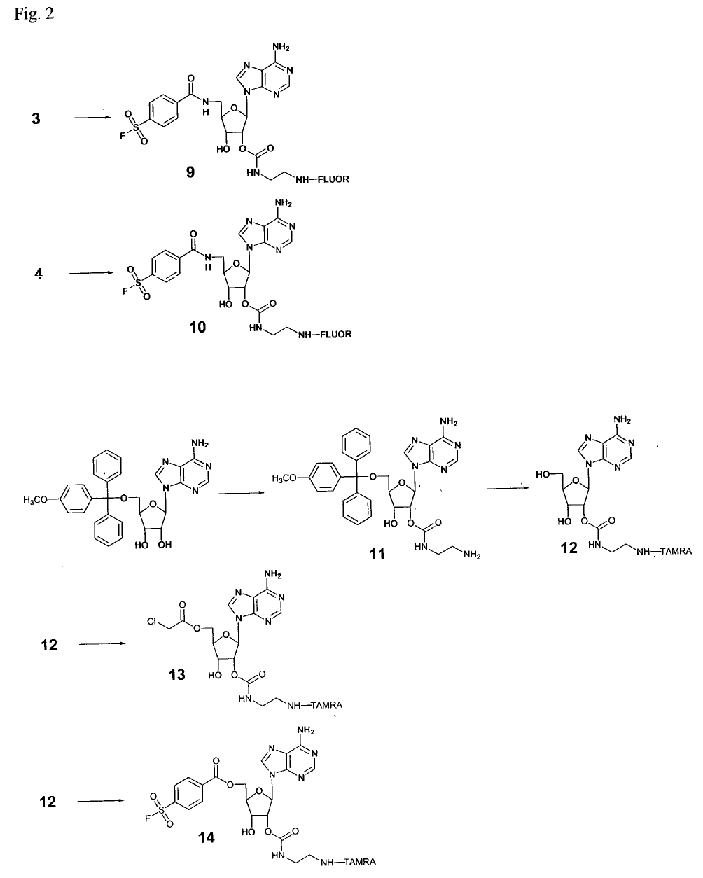

### DETAILED DESCRIPTION OF THE INVENTION

The subject methods and compositions provide enhanced simplicity and accuracy in identifying changes in the presence, amount, or activity of adenine nucleotide-binding proteins in a complex protein mixture, preferably kinases, and most preferably active forms of kinases, using ANBPs that bind to target adenine nucleotide-binding protein(s). The profiling methods described herein can have a number of steps leading to the identification of target adenine nucleotide-binding protein(s) in a complex protein mixture. A complex protein mixture, and preferably two or more complex protein mixtures, e.g., a sample and a control, can be used as obtained from a natural source or as processed, e.g., to remove interfering components and/or enrich the target protein components. Each complex protein mixture to be analyzed is combined under reaction conditions with at least one ANBP to produce conjugates with target adenine nucleotide-binding protein(s). The ANBPs used in two or more complex protein mixtures can differ as to the choice of TAG moiety and/or isotopic composition in order for the labeled complex protein mixtures to be directly compared (e.g., in the same capillary of a capillary electrophoresis apparatus or lane in an electrophoresis gel, or in a mass spectrometer).

The analysis platforms described herein can differ as to the methods of enrichment and analysis using liquid chromatography and/or electrophoresis, and/or mass spectrometry for identification and quantitation. The choice of the platform is affected by the size of the sample, the rate of throughput of the samples, the mode of identification, and the need for and level of quantitation.

Numerous proteins that bind various adenine nucleotides, such as ATP, ADP, AMP, cyclic AMP, etc., are known to those of skill in the art. Of particular interest as target adenine nucleotide-binding proteins in the present invention are protein kinases. Protein kinases are the enzymes responsible for catalyzing the transfer of a γ-phosphoryl group from ATP to the hydroxyl group of serine, threonine or tyrosine residues in peptides, polypeptides, and proteins in a process known as “phosphorylation.”

Protein kinases have been identified in both prokaryotes and eukaryotes, and in both plants and animals. The list of identified kinases is extensive, including the following families of proteins: cyclic nucleotide regulated protein kinase (PKA & PKG) family; diacylglycerol-activated/phospholipid-dependent protein kinase C (PKC) family; kinases that phoshorylate G protein-coupled receptors family; budding yeast AGC-related protein kinase family; kinases that phosphorylate ribosomal protein S6 family; budding yeast DBF2/20 family; flowering plant PVPK1 protein kinase homolog family; kinases regulated by Ca²⁺/CaM and close relatives family; KIN1/SNF1/Nim1 family; cyclin-dependent kinases (CDKs) and close relatives family; ERK (MAP) kinase family; glycogen synthase kinase 3 (GSK3) family; casein kinase II family; Clk family; Src family; Tec/Atk family; Csk family; Fes (Fps) family; Abl family; Syk/ZAP70 family; Tyk2/Jak1 family; Ack family; focal adhesion kinase (Fak) family; epidermal growth factor receptor family; Eph/Elk/Eck receptor family; Axl family; Tie/Tek family; platelet-derived growth factor receptor family; fibroblast growth factor receptor family; insulin receptor family; LTK/ALK family; Ros/Sevenless family; Trk/Ror family; DDR/TKT family; hepatocyte growth factor receptor family, nematode Kin15/16 family; Polo family; MEK/STE7 family; PAK/STE20 family; MEKK/STE11 family; NimA family; wee1/mik1 family; kinases involved in transcriptional control family; Raf family; activin/TGFb receptor family; flowering plant putative receptor kinases and close relatives family; PSK/PTK “mixed lineage” leucine zipper domain family; casein kinase I family; and PKN prokaryotic protein kinase family.

The compositions and methods described herein find use for the most part with biological samples, which may have been subject to processing before reaction with the ANBPs. “Biological sample” intends a sample obtained from a cell, tissue, or organism. Examples of biological samples include proteins obtained from cells (e.g., mammalian cells, bacterial cells, cultured cells, human cells, plant cells, etc.), particularly as a lysate, a biological fluid, such as blood, plasma, serum, urine, bile, saliva, tears, cerebrospinal fluid, aqueous or vitreous humor, or any bodily secretion), a transudate or exudate (e.g. fluid obtained from an abscess or other site of infection or inflammation), a fluid obtained from a joint (e.g. a normal joint or a joint affected by disease such as rheumatoid arthritis, osteoarthritis, gout or septic arthritis), or the like.

Biological samples may be obtained from any organ or tissue (including a biopsy or autopsy specimen) or may comprise cells (including primary cells, passaged or cultured primary cells, cell lines, cells conditioned by a specific medium) or medium conditioned by cells. In preferred embodiments, a biological sample is free of intact cells. If desired, the biological sample may be subjected to prior processing, such as lysis, extraction, subcellular fractionation, and the like. See, Deutscher (ed.), 1990, Methods in Enzymology, vol. 182, pp. 147-238.

Of particular interest are samples that are “complex protein mixtures.” As used herein, this phrase refers to protein mixtures having at least about 20, more usually at least about 50, even 100 or more different proteins, where the particular distribution of proteins is of interest. An example of such a complex protein mixture is a proteome, as defined hereinafter. Complex protein mixtures may be obtained from cells that are normal or abnormal in some particular, where the abnormality is informative as to treatment, status, disease, or the like, can be analyzed using the methods of the subject invention.

The term “proteome” as used herein refers to a complex protein mixture obtained from a biological sample. Preferred proteomes comprise at least about 5% of the total repertoire of proteins present in a biological sample (e.g., the cells, tissue, organ, or organism from which a lysate is obtained; the serum or plasma, etc.), preferably at least about 10%, more preferably at least about 25%, even more preferably about 75%, and generally 90% or more, up to and including the entire repertoire of proteins obtainable from the biological sample. Thus the proteome may be obtained from an intact cell, a lysate, a microsomal fraction, an organelle, a partially extracted lysate, biological fluid, and the like. The proteome will be a mixture of proteins, generally having at least about 20 different proteins, usually at least about 50 different proteins and in most cases 100 different proteins or more.

Generally, the sample will have at least about 1×10¹¹ g of protein, and may have 1 g of protein or more, preferably at a concentration in the range of about 0.1-50 mg/ml. For screening applications, the sample will typically be between about 1×10⁻¹¹ g and about 1×10⁻³ g of protein, preferably between about 1×10⁻⁶ g and 1×10⁻⁴ g of protein. For identification of labeled active target kinases, the sample will typically be between about 1×10⁻⁹ g and about 1 g of protein, preferably between about 1×10⁻⁴ g and 1×10⁻¹ g of protein. The term “about” in this context refers to +/−10% of the amount listed.

The sample may be adjusted to the appropriate buffer concentration and pH, if desired. One or more ANBPs may then be added, each at a concentration in the range of about 1 nM to 20 mM, preferably 10 nM to 1 mM, most preferably 10 nm to 100 μM. After incubating the reaction mixture, generally for a time for the reaction to go substantially to completion, generally for about 0.11-60 minutes, at a temperature in the range of about 5-40° C., preferably about 10° C. to about 30° C., most preferably about 20° C., the reaction may be quenched.

In one aspect of the invention, the method provides for quantitative measurement of target adenine nucleotide-binding protein(s) in biological fluids, cells or tissues. Moreover, the same general strategy can be broadened to achieve the proteome-wide, qualitative and quantitative analysis of target adenine nucleotide-binding protein(s), by employing ANBPs with differing target specificities. The methods and compositions of this invention can be used to identify adenine nucleotide-binding protein(s) of low abundance that are present in complex protein mixtures and can be used to selectively analyze specific groups or classes of adenine nucleotide-binding proteins, such as membrane or cell surface kinases, or kinases contained within organelles, sub-cellular fractions, or biochemical fractions such as immunoprecipitates. Further, these methods can be applied to analyze differences in expressed adenine nucleotide-binding proteins in different cell states. For example, the methods and reagents herein can be employed in diagnostic assays for the detection of the presence or the absence of one or more adenine nucleotide-binding proteins indicative of a disease state, such as cancer.

The subject methods can be used for a variety of purposes. The method can be used in the diagnosis of disease, the response of cells to an external agent, e.g. a drug, staging diseases, such as neoplasia, identifying cell differentiation and maturation, identifying new proteins, screening for active drugs, determining side effects of drugs, identifying allelic response, identifying useful probes from combinatorial libraries, etc.

The system uses ANBPs specific for an adenine nucleotide-binding protein or a group of adenine nucleotide-binding proteins, usually directed to an active site on such proteins, and combines one or a mixture of ANBPs, depending on the specificity of the ANBPs and the variety in the group or groups of proteins to be assayed. In the present invention, it is not necessary that there be no reaction of an ANBP with non-target adenine nucleotide-binding protein(s). Rather, an ANBP is defined as being “specific for,” as “specifically reacting with,” or as “specifically binding to,” target adenine nucleotide-binding protein(s) if the ANBP provides at least about twice the amount of signal from ANBP labeling of target adenine nucleotide-binding protein(s) when compared to an equivalent amount of non-target protein. Preferably the signal obtained from target adenine nucleotide-binding protein(s) will be at least about five fold, preferably 10 fold, more preferably 25-fold, even more preferably 50-fold, and most preferably 100-fold or more, greater than that obtained from an equivalent amount of non-target protein.

The term “target adenine nucleotide-binding protein” as used herein refers to one or more adenine nucleotide-binding protein(s), an adenine nucleotide-binding site of which becomes labeled by one or more ANBPs. Preferred targets are kinases generally classified under the Enzyme Commission number 2.7.1.X. Particularly preferred kinases are protein kinases, classified under the Enzyme Commission number 2.7.1.37. Particularly preferred target kinases include phosphorylase b kinase kinase; glycogen synthase a kinase; hydroxyalkyl-protein kinase; serine(threonine) protein kinase; A-kinase; AP50 kinase; ATP-protein transphosphorylase; bIIPKC; b-andrenergic receptor kinase; calcium/phospholipid-dependent protein kinase; calcium-dependent protein kinase C; cAMP-dependent protein kinase A; cAMP-dependent protein kinase; casein kinase; casein kinase I; casein kinase II; casein kinase 2; cGMP-dependent protein kinase; CK-2; CKI; CKII; cyclic monophosphate-dependent protein kinase; cyclic AMP-dependent protein kinase; cyclic AMP-dependent protein kinase A; cyclic nucleotide-dependent protein kinase; cyclin-dependent kinase; cytidine 3′,5′-cyclic monophosphate-responsive protein kinase; e PKC; glycogen synthase kinase; Hpr kinase; hydroxyalkyl-protein kinase; protein kinase (phosphorylating); casein kinase (phosphorylating); MAPK; mitogen-activated protein kinase; mitogen-activated S6 kinase; M phase-specific cdc2 kinase; p82 kinase; phosphorylase b kinase kinase; PKA; PKC; protein serine kinase; protein kinase A; protein kinase p58; protein phosphokinase; protein glutamyl kinase; protein serine-threonine kinase; protein kinase CK2; protein-aspartyl kinase; protein-cysteine kinase protein-serine kinase; Raf kinase; Raf-1; ribosomal S6 protein kinase; ribosomal protein S6 kinase II; serine kinase; serine-specific protein kinase; serine protein kinase; serine/threonine protein kinase; T-antigen kinase; threonine-specific protein kinase; twitchin kinase; and type-2 casein kinase.

The term “active target adenine nucleotide-binding protein” refers to a target adenine nucleotide-binding protein that is in its native conformation and is able to interact with an adenine nucleotide with which it normally interacts. In effect, the protein is in the form in which it can carry out its biological function.

The term “inactivated” as used herein refers to a sample that has been treated so that at least a portion of target adenine nucleotide-binding protein(s) that were active in the original sample are rendered inactive. An “inactive adenine nucleotide-binding protein” can result from various mechanisms such as denaturation, inhibitor binding, either covalently or non-covalently, mutation, secondary processing, e.g. phosphorylation or dephosphorylation, etc. Functional states of proteins or kinases as described herein may be distinct from the level of abundance of the same proteins or enzymes.

The term “untreated” as used herein refers to a sample that has not been exposed to one or more conditions as compared to a second sample not exposed to such conditions. An untreated sample may be a sample that has not been inactivated; alternatively, an untreated sample may be one not exposed to one or more molecules (e.g., drug lead compounds) in a screening assay. Thus the compositions and methods described herein may comprise comparing a complex protein mixture obtained from cell(s), tissue(s), or organism(s) treated with one or more compounds (e.g., lead compounds in drug discovery) to a complex protein mixture obtained from cell(s), tissue(s), or organism(s) not so treated. ANBP-labeled proteins and/or peptides from the two samples may be compared for relative signal intensity. Such methods may indicate alterations in active protein content due to the treatment regimen. Additionally, such methods can also differentiate between treatments that act by direct inhibition of specific proteins (“primary effects”) versus treatments that affect active protein content upstream, e.g., by altering expression of protein(s) (“secondary effects”).

An “active site” of a protein refers to an area on the surface of a protein, e.g., an enzyme molecule or surface membrane receptor, to which a binding molecule, e.g. substrate or reciprocal ligand, is bound and results in a change in the protein and/or ligand. For a receptor, the conformation may change, the protein may become susceptible to phosphorylation or dephosphorylation or other processing. For the most part, the active site will be the site(s) of an enzyme where the substrate and/or a cofactor bind, where the substrate and cofactor undergo a catalytic reaction; where two proteins form a complex, e.g. the site at which a G protein binds to a surface membrane receptor, two kringle structures bind, sites at which transcription factors bind to other proteins; or sites at which proteins bind to specific nucleic acid sequences, etc.

Structure of ANBPs

The term “adenine nucleotide-binding protein-directed affinity probes” (“ANBPs”) refer to molecules that specifically react with target adenine nucleotide-binding proteins as compared to inactive or non-target proteins. ANBPs may be designed and synthesized using combinatorial chemistry and/or rational design methods. A detailed description of a design strategy that can be adapted to the preparation of ANBPs in which a fluorescent moiety can act as a TAG is provided in PCT Application No. PCT/US02/03808, entitled “Activity Based Probe Analysis” (Attorney Docket No. 063391-0202), filed Feb. 5, 2002, PCT Application No. PCT/US00/34187, WO 01/77684, entitled “Proteomic Analysis,” and PCT Application No. PCT/US00/34167, WO 01/77668, entitled “Proteomic Analysis,” each of which is hereby incorporated by reference in its entirety, including all tables, figures, and claims. Goals of a design strategy are to provide ANBPs that are able to react covalently with a targeted group of adenine nucleotide-binding protein(s), while minimizing non-specific labeling.

The ANBPs of the present invention comprise a warhead linked to a tag by a linker moiety. As will be described hereinafter, each of the warhead, the linker moiety (“L”), and the tag (“TAG”) may be independently selected to provide different target specificities. Each of these components of an ANBP is described in additional detail below.

The term “warhead” as used herein refers to the portion of an ANBP that is directed to, and binds with, an active site of a target adenine nucleotide-binding protein. The warhead comprises a reactive group (“RG”) and an affinity moiety, which is preferably adenine or an analogue thereof. Reactive group (RG) refers to one or more chemical groups within an ANBP that specifically and covalently bond to the active site of a target adenine nucleotide-binding protein.

The term “linker moiety” refers to a bond or chain of atoms used to link one moiety to another, serving as a covalent linkage between two or more moieties. Since in many cases, the synthetic strategy will be able to include a functionalized site for linking, the functionality can be taken advantage of in choosing the linking moiety. The choice of linker moiety has been shown to alter the specificity of targeted probes. See, e.g., Kidd et al., Biochemistry (2001) 40: 4005-15. For example, an alkylene linker moiety and a linker moiety comprising a repeating alkyleneoxy structure (polyethylene glycols, or “PEG”), have distinct specificities and provide distinct protein profiles. Thus, one of skill in the art can select the linker moiety of the ANBP in order to provide additional specificity of the ANBP for a particular protein or protein class.

Linker moieties include among others, ethers, polyethers, diamines, ether diamines, polyether diamines, amides, polyamides, polythioethers, disulfides, silyl ethers, alkyl or alkenyl chains (straight chain or branched and portions of which may be cyclic) aryl, diaryl or alkyl-aryl groups, having from 0 to 3 sites of aliphatic unsaturation. While normally amino acids and oligopeptides are not preferred, when used they will normally employ amino acids of from 2-3 carbon atoms, i.e. glycine and alanine. Aryl groups in linker moieties can contain one or more heteroatoms (e.g., N, O or S atoms). The linker moieties, when other than a bond, will have from about 1 to 60 atoms, usually 1 to 30 atoms, where the atoms include C, N, O, S, P, etc., particularly C, N and O, and will generally have from about 1 to 12 carbon atoms and from about 0 to 8, usually 0 to 6 heteroatoms. The number of atoms referred to above are exclusive of hydrogen in referring to the number of atoms in a group, unless indicated otherwise.

Linker moieties may be varied widely depending on their function, including alkyleneoxy and polyalkyleneoxy groups, where alkylene is of from 2-3 carbon atoms, methylene and polymethylene, polyamide, polyester, and the like, where individual monomers will generally be of from 1 to 6, more usually 1 to 4 carbon atoms. The oligomers will generally have from about 1 to 10, more usually 1 to 8 monomeric units. The monomeric units may be amino acids, both naturally occurring and synthetic, oligonucleotides, both naturally occurring and synthetic, condensation polymer monomeric units and combinations thereof.

The term “TAG” as used herein refers to a moiety that can be used to detect and/or capture the ANBP, in combination with any other moieties that are bound strongly to the TAG, so as to be retained in the process of the reaction of the functional group with the target adenine nucleotide-binding protein. The TAG may be added to a warhead or a warhead-linker combination after reaction with the target adenine nucleotide-binding protein, to form the complete ANBP. For this purpose, the warhead or warhead-linker combination can include a chemically reactive moiety, normally not found in proteins, that will react with a reciprocal functionality on the TAG, e.g. vic.-diols with boronic acid, photoactivated groups, such as diazo bisulfites, etc. The TAG portion permits capture of the conjugate of the target adenine nucleotide-binding protein and the ANBP. The TAG may be displaced from the capture reagent by addition of a displacing TAG, which may be free TAG or a derivative of the TAG, or by changing solvent (e.g., solvent type or pH) or temperature or the linker may be cleaved chemically, enzymatically, thermally or photochemically to release the isolated materials (see discussion of the linker moiety, below).

Examples of TAGs include, but are not limited to, detectable labels such as fluorescent moieties and electrochemical labels, biotin, digoxigenin, maltose, oligohistidine, 2,4-dintrobenzene, phenylarsenate, ssDNA, dsDNA, a polypeptide, a metal chelate, and/or a saccharide. Examples of TAGs and their capture reagents also include but are not limited to: dethiobiotin or structurally modified biotin-based reagents, including deiminobiotin, which bind to proteins of the avidin/streptavidin family, which may, for example, be used in the forms of strepavidin-Agarose, oligomeric-avidin-Agarose, or monomeric-avidin-Agarose; any vicinal diols, such as 1,2-dihydroxyethane (HO—CH₂—CH₂—OH), and other 1,2-dihyroxyalkanes including those of cyclic alkanes, e.g., 1,2-dihydroxycyclohexane which bind to an alkyl or aryl boronic acid or boronic acid esters, such as phenyl-B(OH)₂ or hexyl-B(OEthyl)₂ which may be attached via the alkyl or aryl group to a solid support material, such as Agarose; maltose which binds to maltose binding protein (as well as any other sugar/sugar binding protein pair or more generally to any TAG/TAG binding protein pairs that has properties discussed above); a hapten, such as the dinitrophenyl group, to which an antibody can be generated; a TAG which binds to a transition metal, for example, an oligomeric histidine will bind to Ni(II), the transition metal capture reagent may be used in the form of a resin bound chelated transition metal, such as nitrilotriacetic acid-chelated Ni(II) or iminodiacetic acid-chelated Ni(II); glutathione which binds to glutathione-S-transferase. For the most part, the TAGs will be haptens that bind to a naturally occurring receptor, e.g. biotin and avidin, or an antibody or will be a detectable label, that is also a hapten.

One may use chemical affinity resins, e.g. metal chelates, to allow for digestion of proteins on the solid phase resin and facilitate automation. One example of this is the use of immobilized nickel (II) chelates to purify peptides that have six consecutive histidine residues (His-6 tag) (as described in the Invitrogen product brochureProBond™ Resin (Purification) Catalog nos. R801-01, R801-15 Version D 000913 28-0076), which could be adapted to include non-peptidic chemical linkage coupling a series of imidazole-containing moieties. Alternative chemical attachments include phenyldiboronic acids (described in Bergseid, M. et al. Biotechniques (2000) 29(5), 1126-1133), and disulfide reagents (described in Daniel, S M et al., Biotechniques (1998) 24(3), 484-489). Additionally, chemical affinity tags that are useful in combinatorial synthesis could be adapted for modified peptide purification (reviewed in Porco, J A (2000) Comb. Chem. High Throughput Screening 3(2) 93-102

The term “fluorescent moiety” (“Fl”) refers to a TAG that can be excited by electromagnetic radiation, and that emits electromagnetic radiation in response in an amount sufficient to be detected in an assay. The skilled artisan will understand that a fluorescent moiety absorbs and emits over a number of wavelengths, referred to as an “absorbance spectrum” and an “emission spectrum.” A fluorescent moiety will exhibit a peak emission wavelength that is a longer wavelength than its peak absorbance wavelength. The term “peak” refers to the highest point in the absorbance or emission spectrum.

The fluorescent moiety Fl may be varied widely depending upon the protocol to be used, the number of different ANBPs employed in the same assay, whether a single or plurality of lanes are used in the electrophoresis, the availability of excitation, and detection devices, and the like. For the most part, the fluorescent moieties that are employed as TAGs will absorb in the ultraviolet, infrared, and/or most preferably in the visible range and emit in the ultraviolet, infrared, and/or most preferably in the visible range. Absorption will generally be in the range of about 250 to 750 nm and emission will generally be in the range of about 350 to 800 nm. Illustrative fluorescent moieties include xanthene dyes, naphthylamine dyes, coumarins, cyanine dyes and metal chelate dyes, such as fluorescein, rhodamine, rosamine, the BODIPY dyes (FL, TMR, and TR), dansyl, lanthanide cryptates, erbium. terbium and ruthenium chelates, e.g. squarates, and the like. Additionally, in certain embodiments, one or more fluorescent moieties can be energy transfer dyes such as those described in Waggoner et al., U.S. Pat. No. 6,008,373. The literature amply describes methods for linking fluorescent moieties through a wide variety of linker moieties to other groups. The fluorescent moieties that find use will normally be under 2 kDal, usually under 1 kDal.

Preferred fluorescent moieties Fl can include elaborated conjugated pyran molecules, including xanthenes. Such molecules include eosin, erythrosin, fluorescein, Oregon green, and various commercially available Alexa Fluor® dyes (Molecular Probes, Inc.). Structural examples of such dyes include:

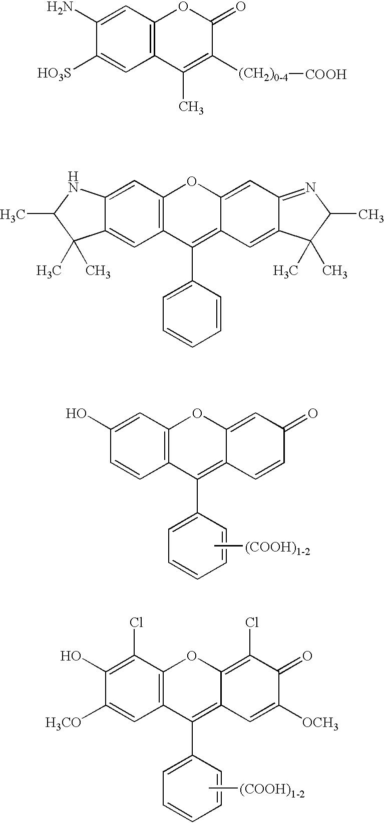

Particularly preferred fluorescent moieties are the rhodamine dyes. These molecules typically have the general structure:

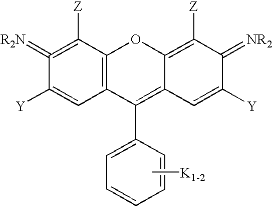

Where K is —CO₂H, or —SO₃H; Y is —H, —CH₃, or together with R forms a six-membered ring; Z is —H or together with R forms a six-membered ring; and R is —H, CH₃, —CH₂CH₃, or together with Y or Z forms a six-membered ring. Rhodamine molecules such as tetramethylrhodamine, 5-carboxytetramethylrhodamine, 6-carboxytetramethylrhodamine, carboxyrhodamine-6G, rhodamine-B sulfonyl chloride, rhodamine-red-X, and carboxy-X-rhodamine are well known to those of skill in the art. See, e.g., Handbook of Fluorescent Probes and Research Products, Molecular Probes, Inc., 2001, which is hereby incorporated by reference in its entirety. Advantageous properties of rhodamines include high quantum yields, low sensitivity of fluorescence over a pH range of from about pH 3 to about pH 8, advantageous water solubility, good photostability, and absorption of light in the visible spectrum. Particularly preferred fluorescers are 5-carboxytetramethylrhodamine and 6-carboxytetramethylrhodamine.

Other preferred fluorescent moieties Fl include the BODIPY dyes, which are elaborations of a 4-bora-3a,4a-diaza-s-indacene structure. Exemplary structures are provided below:

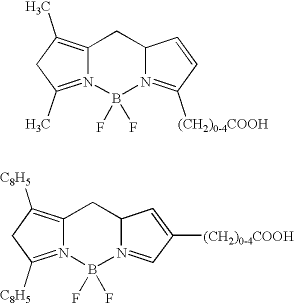

Yet other preferred fluorescent moieties include the cyanine dyes, conjugated structures comprising a polymethine chain terminating in nitrogen atoms. Typically, the nitrogens are themselves part of a conjugated heterocycle. An exemplary structures is provided below:

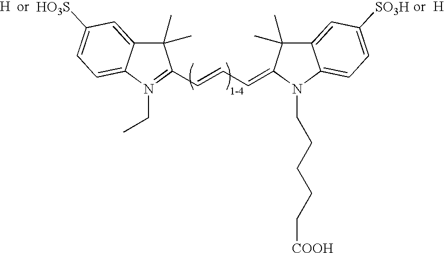

Also of interest for use as TAGs are matched dyes as described in U.S. Pat. No. 6,127,134, which is hereby incorporated by reference in its entirety, including all tables, figures, and claims, which is concerned with labeling proteins with dyes that have different emissions, but have little or no effect on relative migration of labeled proteins in an electrophoretic separation. Of particular interest are the cyanine dyes disclosed therein, being selected in '134 because of their positive charge, which matches the lysine to which the cyanine dyes bind. In addition there is the opportunity to vary the polyene spacer between cyclic ends, while keeping the molecular weight about the same with the introduction of an alkyl group in the shorter polyene chain dye to offset the longer polyene. Also described are the BODIPY dyes, which lack a charge. The advantage of having two dyes that similarly affect the migration of the protein would be present when comparing the native and inactived samples, although this would require that in the inactivated sample at least a portion of the protein is monosubstituted.

In each of the foregoing examples of preferred fluorescent moieties, carboxyl groups can provide convenient attachment sites for linker moieties. In the particularly preferred 5- and 6-carboxyrhodamine molecules, the 5- or 6-carboxyl is particularly preferred as an attachment site:

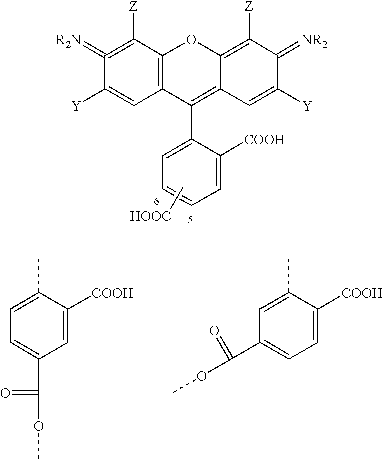

In general, any affinity label-capture reagent commonly used for affinity enrichment, which meets the suitability criteria discussed above, can be used in the method of the invention. Biotin and biotin-based affinity TAGs are particularly illustrated herein. Of particular interest are structurally modified biotins, such as deiminobiotin or dethiobiotin, which will elute from avidin or streptavidin (strept/avidin) columns with biotin or under solvent conditions compatible with ESI-MS analysis, such as dilute acids containing 10-20% organic solvent. For example, deiminobiotin tagged compounds will elute in solvents below about pH 4.

In certain embodiments, ANBPs can be immobilized on a solid phase to form a “tethered” ANBP. In preferred embodiments, a plurality of different ANBPs may be tethered to different regions of one or more solid phases to form a patterned array. Such a patterned array having two or more regions comprising ANBPs that differ in structure and/or reactivities from each other could be used to simultaneously measure the presence, amount, or activity of a plurality of target adenine nucleotide-binding proteins. The term “solid phase” as used herein refers to a wide variety of materials including solids, semi-solids, gels, films, membranes, meshes, felts, composites, particles, and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Diamandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain et al., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001).

The ANBP(s) employed will have an affinity for an active site, which may be specific for a particular active site or generally shared by a plurality of related adenine nucleotide-binding proteins. The affinity may be affected by the choice of the functional group, the linker moiety, the binding moiety, the TAG, or a combination thereof. One or more ANBPs may be designed that exhibit specificity for a single target adenine nucleotide-binding protein, or that exhibit specificity for a plurality of targets that may be structurally or functionally related.

Particularly preferred ANBPs of this invention come within the following formulae:

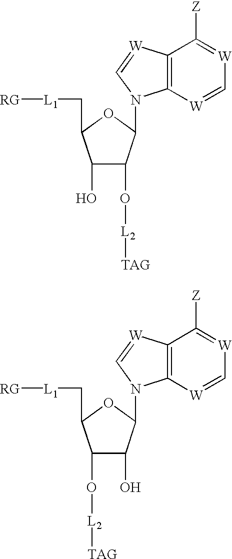

wherein

each W is independently carbon or nitrogen, particularly nitrogen;

Z is hydrogen or amino, particularly amino;

RG is a reactive group capable of reacting with at least one of thiol, hydroxyl, carboxyl or amino joined through L₁ to the 5′ carbon of the ribose, where the groups may be fluorosulfonyl, fluorophosphonyl ester, halogen, epoxide or ethylene α to an activating group, such as sulfonyl, carbonyl, phosphonyl, phosphityl, etc., or halogen β to an activating group, such as amino, thio, etc.;

L₁ and L₂ are optionally present and are independently alkyl or heteroalkyl groups of 1-20 backbone atoms selected from the group consisting of —N(R)—, —O—, —S— or —C(R)(R)—, where each R is independently H or —C₁₋₆ alkyl straight or branched chain. The person of ordinary skill will realize that pharmaceutically acceptable salt or complexes of these compounds are also useful and are also contemplated within the scope of the invention;

TAG is a label joined to the oxygen of the 2′ and/or 3′ position of the ribose through a linking group L₂ as defined above,

where the entire molecule will generally have not more than about 75 carbon atoms and at least about 15 carbon atoms, usually at least about 20 carbon atoms, there being at least about 8 heteroatoms, which will generally include halogen, oxygen, sulfur, nitrogen and phosphorous.

A preferred reactive group RG will come within the following formulae:

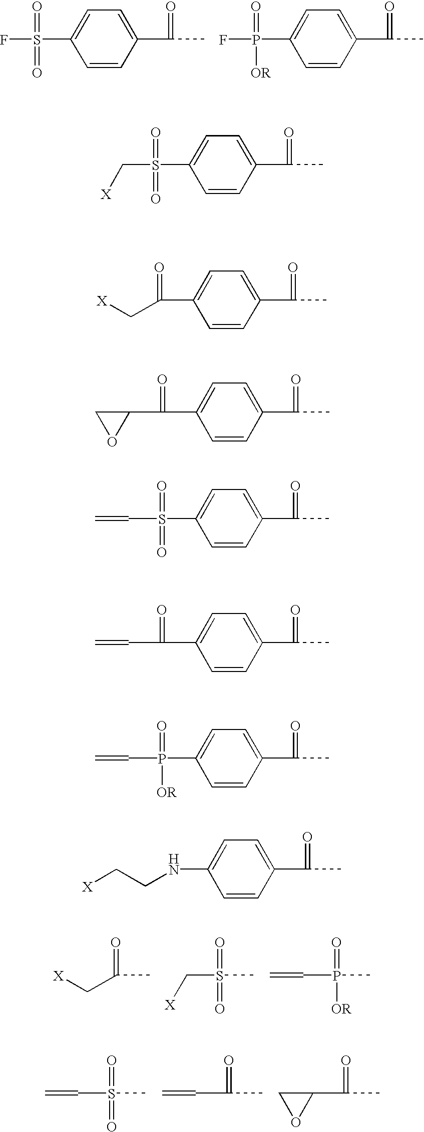

where X is halogen, particularly fluorine, chlorine, bromine or iodine.

A preferred combination of TAG and linking groups (RG) come within the following formulae:

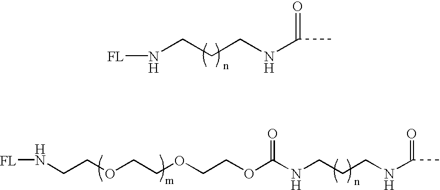

where n and m are independently in the range of 0 to 8 and Fl is a TAG, usually a fluorescer, although it may also be a non-fluorescent label or ligand, e.g. biotin.

The TAGs may be varied widely, generally being organic molecules of from about 150 to 600 Dal and having a natural receptor or an antibody can be prepared. The most common TAG is biotin (including iminobiotin and dethiobiotin), with strept/avidin as the receptor, although there are numerous other compounds for which there are receptors, such as drugs and their target proteins, sugars and lectins, enzymes and substrates, etc. TAGs for which antibodies are available include digoxin, 2,4-dinitrobenzene, phenylarsenate, etc. The TAG will be selected for binding affinity, non-interference, availability, and the like, and may include fluorescers, where the fluorescer will serve both functions as ligand and detectable fluorescent label.

The TAGs, fluorescers and ligands, of particular interest come within the following formulae:

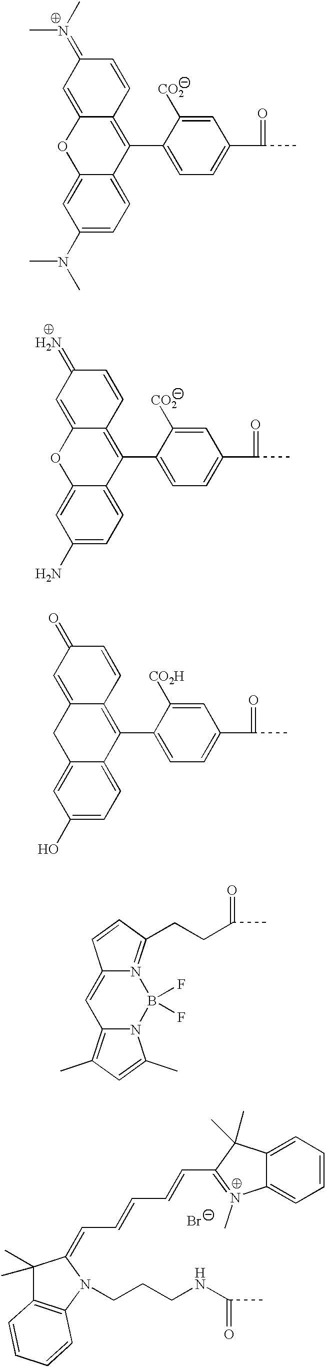

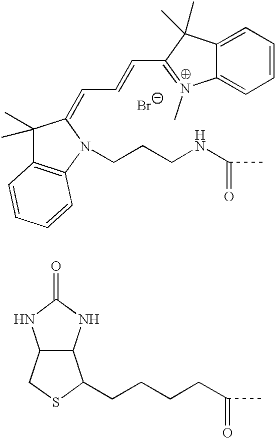

The compounds of the subject invention may be prepared in accordance with conventional methods. For introducing the TAG, one can derivatize adenosine or 5′-amino adenosine at the 2′ or 3′ position using conventional reagents. For example, the amino adenosine may be combined with a dicarbonate diester, followed by the addition of a carbodiimide and a diamine to provide a product having an available amino group for conjugation. A fluorescent compound having an active carboxyl may then be used to form the amide. After deprotection of the 5′-amino group, the reactant having the reactive group as well as a functionality for coupling to the amino group, e.g. activated carboxyl, is added to provide the final product. An analogous process may be used with a 5′-hydroxyl, where the 5′-hydroxyl is protected, followed by functionalization of the 2′ or 3′ hydroxyl as above. The process is then repeated in substantially the same manner.

Analysis of Samples With ANBPs

After the reaction between the complex protein mixture and the ANBP(s) is completed, the conjugates of the probes and protein targets will be analyzed. Preferably, the ANBPs of the present invention comprise a TAG that allows for manipulation of the conjugates, either for sequestering the conjugates or detecting the conjugates or both. The probes may be analyzed by separating into components, e.g., by electrophoresis, for example gel electrophoresis, capillary electrophoresis or microfluidic electrophoresis; mass spectrometry, e.g., MALDI-TOF, microcapillary liquid chromatography-electrospray tandem MS, or other technique. To enhance the analysis, the conjugates may be deglycosylated using an appropriate glycosidase, such as PGNaseF, under conventional deglycosylation conditions indicated by the enzyme supplier. Labeled active target proteins can be identified based on a variety of physical criteria, such as apparent molecular weight, peptide sequence composition, enzymatic activity (e.g., serine hydrolase activity), or a combination of such criteria. Suitable analysis methods are disclosed in, e.g., PCT Application No. PCT/US02/03808, entitled “Activity Based Probe Analysis” (Attorney Docket No. 063391-0202), filed Feb. 5, 2002, PCT Application No. PCT/US00/34187, WO 01/77684, entitled “Proteomic Analysis,” and PCT Application No. PCT/US00/34167, WO 01/77668, entitled “Proteomic Analysis,” each of which is hereby incorporated by reference in its entirety, including all tables, figures, and claims.

The term “separating” as used herein refers to methods that enrich the concentration of a molecule of interest in a particular location or container relative to other molecules originally present. For example, gel electrophoresis enriches the concentration of molecules that migrate at a particular rate relative to other molecules originally present that migrate at different rates; sequestration methods enrich the concentration of molecules capable of being sequestered (e.g., by binding to a receptor) relative to other molecules not so capable (e.g., removed by washing out molecules that do not bind to a receptor). Numerous additional analytical procedures are known to the artisan for separating and analyzing complex protein mixtures (e.g., chromatographic methods such as HPLC, FPLC, ion exchange, size exclusion; mass spectrometry; differential centrifugation).

In preferred embodiments, the probe products are analyzed by electrophoresis, e.g., slab gel, capillary or microfluidic, optionally using a gel for separation of the different components. In particularly preferred embodiments, SDS-PAGE is used, including 2D PAGE. The sample composition may be preliminarily separated using isoelectric focusing, followed by using bands or regions for further electrophoretic separation. Conventional conditions can be employed for the electrophoresis, using a denaturing medium, so that the active sample and the inactivated sample are both denatured in the gel. Numerous patents have issued for performing electrophoresis for the separation of proteins. See, e.g., U.S. Pat. Nos. 4,415,655; 4,481,094; 4,865,707; and 4,946,794. Texts describing procedures include Laemmli, Nature 227:680-685 (1970); Sambrook et al., “Molecular Cloning: A Laboratory Manual.” 3^{rd} Edition, Cold Spring Harbor Press, Cold Spring Harbor, N.Y. (2001).

Using the ANBPs of the present invention, labeled target adenine nucleotide-binding protein(s) may be identified by excitation and detection of light emitted upon excitation of the fluorescent moiety, e.g., in electrophoresis gels. In certain embodiments, such as when the ANBP(s) label(s) a plurality of target adenine nucleotide-binding proteins or when the identity of a labeled target adenine nucleotide-binding protein is unknown, the labeled target adenine nucleotide-binding protein(s) present in various electophoretic bands may be further assayed to identify the specific proteins to which the ANBP(s) bound, e.g., by fragmentation and mass spectrometric analysis. In particular, the sequence of proteins can be determined using tandem MS (MSⁿ), techniques. By application of sequence database searching techniques, the protein from which a sequenced peptide originated can be identified.

In designing a gel-based analysis system, the artisan may balance various considerations, such as speed, resolution, sample volume, choice of fluorophore, detection methods, etc., in order to arrive at an optimal solution. For example, for simple screening analysis (i.e., when gel bands are not to be identified by means of eluting proteins from the gel matrix for further analysis), very thin gels may be run quickly. Additionally, such thin gels are amenable to the use of laser-induced fluorescence scanning systems and narrow gel lanes, as laser focusing and confocal detection optics permit the detection of very small amounts of ANBP in a sample. Conversely, thicker gels may be advantageous in protein identification analysis, as a sufficient amount of material must be obtained from a gel band to permit further manipulations.

For rapid screening analysis, a suitable gel electrophoresis platform would consist of a glass sandwich gel format of from 15-40 cm in width, 20-40 cm in length, and from 0.6 to 0.2 cm in thickness. A particularly preferred format is from about 30-35 cm in width, about 25-30 cm in length, and about 0.4 mm in thickness. The term “about” in this context refers to +/−10% of a given dimension. The gel format is preferably combined with a laser-induced fluorescence detector apparatus comprising detection optics that permit sampling of the gel without removal from the gel plates, as such thin gels may be extremely fragile. Typically, such an instrument uses confocal optics for detection. By matching the thickness of the gel to the thickness of the confocal “slice,” signal detection can be matched to a minimal amount of sample.

The spacing between sample wells is limited only by the amount of sample necessary to obtain a sufficient signal for measurement. Appropriate spacings are between 1 and 4 mm, most preferably about 2.25-3 mm. The term “about” in this context refers to +/−10% of the spacing between wells. Selecting a spacing between wells of about 2.25 mm as an example, a gel platform 25 cm in width could accommodate as many as 96 individual samples.

After completing the electropherogram, the bands may then be read using any convenient detection means (e.g., a fluorescent reader, e.g., Hitachi FMbio Flatbed Fluorescence Scanner, when the ANBP comprises a fluorescent moiety), where the intensity of each band may be transferred to a data processor for processing. Depending on whether one or more lanes are involved with the analysis, the data may be compiled from a single or multiple lanes to establish the bands associated with target adenine nucleotide-binding proteins that are absent from a particular sample, the different target adenine nucleotide-binding proteins that reacted with different probes as evidenced by the different fluorescence emission for each of the probes, and any cross-reactivity between the probes. The bands that are obtained in the gel are sharp and provide for excellent resolution. Particularly, much better resolution and sensitivity may be obtained than when biotin-labeled probes are used, followed by complex formation with labeled avidin, and Western blotting.

The results obtained from analyzing the adenine nucleotide-binding protein profiles may then be organized in a manner that allows for ready comparisons and differentiation between samples. One technique that finds utility is cluster analysis. One applies a hierarchical clustering algorithm to the samples using the Pearson correlation coefficient as the measure of similarity and average linking clustering (Cluster program: Ross et al., Nat. Genet. 24:227-35 (2000), Eisen et al., Proc. Natl. Acad. Sci. USA 95:14863-68 (1998)). For each labeled adenine nucleotide-binding protein, averaged cell sample values are compared to identify the cell sample that expressed the highest level of a particular protein. The levels may then be expressed as a percentage of this highest level to normalize the data sets. As data sets are built up from cell samples, the cluster analysis can be modified in light of new data that provides a new maximum for a particular protein, so that one may have cluster analysis within a given group of samples as well as cluster analysis extending over many samples and groups of samples. Cluster analysis can also be applied as to the individual fractions and pair-wise combinations, so as to maximize information from the cell samples in relating the samples to each other and standards. For large numbers of samples, clustergrams can be used to rapidly identify the similarities between samples, for example, in terms of origin of the cells, aggressiveness and invasiveness, diagnosis, prognosis, preferential therapies and how the tumor has responded to a course of treatment.

Following ANBP labeling of target adenine nucleotide-binding protein(s), protein digestion may be employed to produce both unlabeled and ANBP-labeled peptides. The digestion may be performed while the proteins are in solution or when the conjugates are sequestered, e.g., by receptors bound to a solid support. Digestion preferably employs only one protease; however, two or more, usually not more than three, proteases may be used. The proteases may be in solution or bound to a surface. The proteases may be combined in the same reaction mixture, or the sample may be divided into aliquots and each of the aliquots treated with a different protease. Digestion may also occur before binding to the conjugate to a support and/or a after the conjugates are bound to a solid support. Enzymes that find use include, but are not limited to, trypsin, chymotrypsin, bromelain, papain, carboxypeptidase A, B and Y, proteinase A and K, chymopapain, plasmin, subtilisin, clostripain etc.

In particularly preferred embodiments, additional steps can be used to reduce the complexity of the analysis to be performed. For example, the complex protein mixture can be denatured following labeling, e.g., by the addition of urea, guanidinium salts, detergents, organic solvents, etc., in order to reduce or eliminate unwanted proteolysis from endogenous proteases present in the mixture. Additionally, cysteine residues can be reduced and alkylated to maintain the homogeneity of cysteine-containing peptides and to prevent refolding of endogenous proteases following removal of the denaturant. Moreover, proteases can be combined with additional enzymes, such as glycosidases, phosphatases, sulfatases, etc., that can act to remove post-translational modifications from proteins. Examples of such post-translational modifications include, but are not limited to, glycosylations, phosphorylations, sulfations, prenylations, methylations, amidations, and myristolations. Such steps can be mixed and matched by the skilled artisan, depending on the requirements of a particular analysis.

Prior to digestion, a buffer exchange step may be employed, e.g., by gel filtration, dialysis, etc. This step may be used to remove excess ANBPs, to remove denaturant, and/or to provide suitable buffer conditions for digestion. In particularly preferred embodiments, buffer exchange is performed by gravity flow gel filtration.

Digestion will be carried out in an aqueous buffered medium, generally at a pH in the range of about 4 to 10, depending on the requirements of the protease. The concentration of the protease will generally be in the range of about 6×10⁻⁸ M to about 6×10⁻⁶ M, more preferably in the range of about 1.8×10⁻⁸ M to about 2×10⁻⁷ M, and most preferably about 6×10⁻⁷ M (e.g., 150 ng/10 μL). The term “about” in this context means +/−10% of a givem measurement. The time for the digestion will be sufficient to go to at least substantial completion, so that at least substantially all of the protein will have been digested. Digests may be performed at a temperature that is compatible with the protease(s) employed, preferably from 20° C. to 40° C., most preferably about 37° C. Where the digestion takes place in solution, the protease may be quenched by any convenient means, including heating or acidification of the sample. Alternatively, quenching can be achieved by sequestering the fragment conjugates with a receptor for the TAG bound to a surface, or by addition of a protease inhibitor (e.g., E64, DIFP, PMSF, etc.). Where the proteins are bound to a surface, the proteases may be washed away before the bound digested protein is released.

Following protein digestion, peptides can be sequestered, e.g., by binding to receptors for the TAG of one or more ANBP-labeled peptides. Preferably, sequestration relies on receptors bound to a solid support that can be easily manipulated during wash steps. The support may be beads, including paramagnetic beads, prepared from various materials, such as Bioglas, polystyrene, polyacrylate, polymethylmethacrylate, polyethylene, polysaccharides, such as Agarose, cellulose, amylose, etc., polyurethane, and the like. Desirably, the support surface will not interfere with the binding of TAG to its cognate receptor, and the receptor may be linked to the support by a hydrophilic bridge that allows for the receptor to be removed from the surface. When beads are employed, the beads will generally have a cross-dimension in the range of about 5 to 100μ.Instead of beads, one may use solid supports, such as slides, the walls of vessels, e.g. microtiter well walls, capillaries, etc. There is an extensive literature of receptor bound supports that is readily applicable to this invention, since the sequestering step is conventional. The sample is contacted with the support for sufficient time, usually about 5 to 60 min, to allow all of the conjugate to become bound to the surface. At this time, all of the non-specifically bound components from the sample may be washed away, greatly enriching the target proteins as compared to the rest of the sample.

Following separation by sequestration, ANBP-labeled peptides may then be released from the receptor. The particular method of release will depend upon the TAG-receptor pair. In some instances, one may use an analog of the TAG as a “releasing agent” to release the conjugate. This is illustrated by the use of deimino- or dethiobiotin as the TAG and biotin as the releasing agent. Where this is not convenient, as in the case of many fluorescent moieties as TAGs where there may not be a convenient analog, conditions such as high salt concentrations, chaeotropic agents (e.g., isothiocyanate or urea) low pH, detergents, organic solvents, etc., may be used to effect release. Once the conjugate has been released, dialysis, ion exchange resins, precipitation, or the like may be used to prepare the conjugate solution for the next stage.

Where the migration rates in various separation procedures provide the necessary identification of the peptide(s) generated and, therefore, the protein from which they are obtained, no further analysis may be required. However, where further identification is desired or the earlier results do not provide certainty as to the identification and amount of a particular component, an identification method using mass spectrometry (MS) can be employed. See, for example, WO 00/11208. The use of mass spectrometry will be described below. Such identification methods potentially provide greater information, but requires greater sample size in comparison to, for example, capillary electrohoresis, and has a lower throughput.

Chromatographic and/or electrophoretic separation methods as described herein may be used to simplify the mixtures introduced into the mass spectrometer, allowing for a more accurate analysis. For ANBP-labeled peptides, the use of fluorescent moieties as ANBP TAGs can permit the use of an online fluorescence detector to trigger ESI-MS data collection or fraction collection for subsequent analysis, e.g., providing sample on a MALDI plate. In this way, only fractions and bands that contain ANBP-labeled peptides will be selected for further processing, thereby avoiding using the MS with certain fractions.

In particularly preferred embodiments, the identification methods described herein can be combined with one or more separation methods to develop a “separation profile” that can be used to identify peptides without the need for MS analysis. In these methods, a sample (e.g., material from a chromatography column) is divided into at least two portions; one portion is used for MS analysis, and the other portion(s) are used for one or more separation methods (e.g., a single CE run, or two or more CE runs using different separation conditions). The peptide identification obtained from the MS analysis can be assigned to the observed separation profile (e.g., the elution time of the peptide observed in the CE run(s)). Observation of this separation profile in subsequent samples can then be correlated to the peptide known to exhibit that separation profile.

The identification methods described herein may also utilize ANBPs that differ isotopically in order to enhance the information obtained from MS procedures. For example, using automated multistage MS, the mass spectrometer may be operated in a dual mode in which it alternates in successive scans between measuring the relative quantities of peptides obtained from the prior fractionation and recording the sequence information of the peptides. Peptides may be quantified by measuring in the MS mode the relative signal intensities for pairs of peptide ions of identical sequence that are tagged with the isotopically light or heavy forms of the reagent, respectively, and which therefore differ in mass by the mass differential encoded with the ANBP. Peptide sequence information may be automatically generated by selecting peptide ions of a particular mass-to-charge (m/z) ratio for collision-induced dissociation (CID) in the mass spectrometer operating in the MSⁿ mode. (Link, et al., (1997) Electrophoresis 18:1314-34; Gygi, et al., (1999) idid 20:310-9; and Gygi et al., (1999) Mol. Cell. Biol. 19:1720-30). The resulting CID spectra may be then automatically correlated with sequence databases to identify the protein from which the sequenced peptide originated. Combination of the results generated by MS and MSⁿ analyses of affinity tagged and differentially labeled peptide samples allows the determination of the relative quantities as well as the sequence identities of the components of protein mixtures.

Protein identification by MSⁿ may be accomplished by correlating the sequence contained in the CID mass spectrum with one or more sequence databases, e.g., using computer searching algorithms (Eng. et al. (1994) J. Am. Soc. Mass Spectrom. 5:976-89; Mann, et al., (1994) Anal. Chem. 66:4390-99; Qin, et al., (1997) ibid 69:3995-4001; Clauser, et al., (1995) Proc. Natl. Acad. Sci. USA 92:5072-76). Pairs of identical peptides tagged with the light and heavy affinity tagged reagents, respectively (or in analysis of more than two samples, sets of identical tagged peptides in which each set member is differentially isotopically labeled) are chemically identical and therefore serve as mutual internal standards for accurate quantitation. The MS measurement readily differentiates between peptides originating from different samples, representing different cell states or other parameter, because of the difference between isotopically distinct reagents attached to the peptides. The ratios between the intensities of the differing weight components of these pairs or sets of peaks provide an accurate measure of the relative abundance of the peptides and the correlative proteins because the MS intensity response to a given peptide is independent of the isotopic composition of the reagents. The use of isotopically labeled internal standards is standard practice in quantitative mass spectrometry (De Leenheer, et al., (1992) Mass Spectrom. Rev. 11:249-307).

The following examples are offered by way of illustration and not by way of limitation.

### EXAMPLES

### Example 1

### Synthesis of 5′-FSB-amido-2′(3′)-(2-TAMRA-amidoethylcarbamoyl)adenosine (5)

5′-(Boc)-amino-2′(3′)-(2-aminoethylcarbamoyl)adenosine (1)

To 5′-amino adenosine (20 mg, 0.075 mmol) dissolved in DMF (1 ml) was added di-t-butyl dicarbonate (14 mg, 0.075 mmol) and the resulting clear solution stirred at room temperature. After 30 minutes carbonyldiimidazole (24 mg, 0.15 mmol, 2 eq.) was added and the resulting clear solution stirred at room temperature for 3 hours. The reaction was quenched with methanol (0.5 ml) and volatiles removed under high vacuum leaving a white solid. The solid was dissolved in DMF (1 ml), and ethylene diamine (50 μl, 55.6 mg, 0.75 mmol, 10 eq.) added to yield a clear solution that was stirred at room temperature. After 10-minutes, LC-MS confirmed that the reaction was complete (isomers inseparable by LC—one peak) and indicated that 1 (24 mg, 0.053 mmol, 71%) had been formed in purity suitable for the next step. An analytical sample was prepared by preparative HPLC and was analyzed by ¹H-NMR. It was a 65/35 mixture of two isomers having the characteristic nucleoside ¹H chemical shifts: glycosidic CH 5.90 (d, 1H) and 6.12 (d, 1); imidazole CH 8.59 (s, 1H) and 8.63 (s, 1H); pyridazine CH 8.39 (s, 1H) and 8.41 (s, 1H); BOC t-butyl CH₃ 1.36 (s, 9H) and 1.38 (s, 9H) ppm. It had mass spectrum: [MH⁺]=453.2 amu (calculated for C₁₈H₂₈N₈O₆ 452.48 amu).

5′-Amino-2′(3′)-(2-TAMRA-amidoethylcarbamoyl)adenosine (2)

At room temperature, 5′-(Boc)-amino-2′(3′)-(2-aminoethylcarbamoyl)adenosine 1 (8 mg, 0.018 mmol) was dissolved in DMF (0.5 ml) followed by addition of TAMRA-SE (10 mg, 0.019 mmol, 1.05 eq.) dissolved in DMF (1 ml). After 30 minutes (2 isomers now separable by LC) volatiles were removed under high-vacuum leaving a red residue. The residue was dissolved in TFA (1 ml) for 1-minute, then concentrated under high vacuum. LC-MS shows completion of reaction and again two isomers were observed. Preparative HPLC (the most abundant and easily separated of the two isomers was harvested [this isomer had the longest retention time of the two on a C8 reverse phase column]) yielded 2 as a TFA salt (8.5 mg, 0.010 mmol, 54%). It had mass spectrum: [MH⁺]=765.3 amu (calculated for C₃₈H₄₀N₁₀O₈ 764.80 amu).

5′-Amino-2′(3 ′)-(2-BODIPY-FL-amidoethylcarbamoyl)adenosine (3)

The BODIPY-FL analogue 3 was prepared in exactly the same manner as 2 except that BODIPY-FL succinimidyl ester was used in place of TAMRA succinimidyl ester. The isomeric products were separated similarly on HPLC with the most abundant isomer taken on to the next step. It had mass spectrum: [MH⁺]=627.3 amu (calculated for C₂₇H₃₃BF₂N₁₀O₅ 626.43 amu).

5′-Amino-2′(3′-(2-rhodamine Green-amidoethylcarbamoyl)adenosine (4)

The rhodamine green analogue 4 was prepared in exactly the same manner as 2 except that rhodamine green succinimidyl ester was used in place of TAMRA succinimidyl ester. The isomeric products were separated on HPLC with the most abundant being taken on to the next step. It had mass spectrum: [MH⁺]=709.3 amu (calculated for C₃₄H₃₂N₁₀O₈ 708.69 amu).

5′-FSB-amido-2′(3′)-(2-TAMRA-amidoethylcarbamoyl)adenosine (5)

To 5′-Amino-2′(3′)-(2-TAMRA-amidoethylcarbamoyl)adenosine (2) (2 mg, 2.62 μmol, [TFA salt]) dissolved in DMF (1 ml) was added 4-fluorosulfonylbenzoylchloride (3 mg, 40.4 μmol, 20 eq.). Triethylamine was then added portion wise (3×5 μl) over 15 minutes at room temperature. LC-MS showed a mono-adduct as the major product among several by-products. The compound was isolated by preparative HPLC to yield 5 (1.9 mg, 1.78 μmol, 70%). It had mass spectrum: [MH⁺] 951.3 amu (calculated for C₄₅H₄₃FN₁₀O₁₁S 950.96 amu).

### Example 2

### Synthesis of 5′-(4″-Vinylsulfonylbenzoyl)amido-2′(3′-(2-TAMRA amidoethylcarbamoyl)adenosine (6)

The 5′-vinylsulfonylbenzoyl probe 6 was prepared in the same manner as 5, however, succinimidyl-(4-vinylsulfonyl)benzoate was used in place of 4-fluorosulfonyl-benzoylchloride. Preparative HPLC yielded 6 in 82% yield. It had mass spectrum: [MH⁺]=959.3 amu (calculated for C₄₇H₄₆N₁₀O₁₁S 959.03 amu).

### Example 3

### Synthesis of 5′-Acrylamido-2′(3 ′)-(2-TAMRA-amidoethylcarbamoyl)adenosine (7)

The 5′-acrylamido probe 7 was prepared in the same manner as 5 however, acryloyl chloride was used in place of 4-fluorosulfonylbenzoylchloride. Preparative HPLC yielded 7 in 75% yield. It had mass spectrum: [MH⁺]=819.3 amu (calculated for C₄₁H₄₂N₁₀O₉ 818.84 amu).

### Example 4

### Synthesis of 5′-α-Chloroacetamido-2′(3′)-(2-TAMRA-amidoethylcarbamoyl)adenosine (8)

The 5′-α-chloroacetamido probe 8 was prepared in the same manner as 5, however, chloroacetyl chloride was used in place of 4-fluorosulfonylbenzoylchloride. Preparative HPLC yielded 8 in 92% yield. It had mass spectrum: [MH⁺]=841.3 amu (calculated for C₄₀H₄₁FN₁₀O₉ 841.28 amu).

### Example 5

### Synthesis of 5′-FSB-amido-2′(3′)-(2-BODIPY-FL-amidoethylcarbamoyl)adenosine (9)

The BODIPY-FL FSBA probe analogue 9 was prepared from 3 using similar conditions to that of 5 with similar HPLC purification to provide 9 in 69% yield. It had mass spectrum: [MH⁺]=813.3 amu (calculated for C₃₄H₃₆BF₃N₁₀O₈S 812.59 amu).

### Example 6

### Synthesis of 5′-FSB-amido-2′(3′)-(2-rhodamine Green-amidoethylcarbamoyl)adenosine (10)

The rhodamine green FSBA probe analogue 10 was prepared from 4 using similar conditions to that of 5 with similar HPLC purification to provide 10 in 79% yield. It had mass spectrum: [MH⁺]=895.2 amu (calculated for C₄₁H₃₅FN₁₀O₁₁S 894.85 amu).

### Example 7

### Synthesis of 5′-α-Chloroacetyl-2′(3′)-(2-TAMRA-amidoethylcarbamoyl)adenosine (13)

5′-Monomethoxytrityl-2′(3′)-(2-aminoethylcarbamoyl)adenosine (11)

5′-Monomethoxyttrityl adenosine (200 mg, 0.37 mmol) was dissolved in DMF (3 ml) and to the resulting clear solution was added carbonyl diimidazole (120 mg, 0.74 mmol, 2 eq.). After 2 hours the reaction was quenched with methanol (0.5 ml). The volatiles were removed under vacuum to leave a clear syrup which was then dissolved in DMF (1 ml) followed by ethylene diamine (80 μl, 1.20 mmol, ca. 4 eq.). LC-MS indicated that the reaction was complete within minutes and the volatiles were removed under vacuum to yield 11 as a viscous clear syrup pure enough for the next step. An analytical sample was purified by preparative HPLC. It was analyzed by ¹H-NMR and was shown to be a 65/35 mixture of isomers (and an unavoidable small amount of detritylated material due to the presence of TFA in the HPLC buffers). The isomers had characteristic nucleoside ¹H chemical shifts: glycosidic CH 5.92 (d, 1H) and 6.09 (d, 1H); imidazole CH 8.23 (s, 1H) and 8.26 (s, 1H); pyridazine CH 8.07 (s, 1H) and 8.09 (s, 1H); methoxy CH₃ 3.72 (s, 3H) and 3.72 (s, 3H) ppm. It had mass spectrum: [MH⁺]=626.3 amu (calculated for C₃₃H₃₅N₇O₆ 625.68 amu).

5′-Hydroxy-2′(3′)-(2-TAMRA-amidoethylcarbamoyl)adenosine (12)

5′-Monomethoxytrityl-2′(3′)-(2-aminoethylcarbamoyl)adenosine 11 (30 mg, 48.0 μmol) was dissolved in DMF (300 μl) and TAMRA succinimidyl ester (25 mg, 48.0 μmol) dissolved in DMF solution (250 μl) added. The resulting red solution was then allowed to stir at room temperature for 30 minutes. LC-MS confirmed completion of reaction. Removal of the volatiles provided a red residue that was then momentarily exposed to neat TFA (orange solution). Removal of the volatiles and purification by preparative HPLC yielded 12 as a clear residue. LC-MS confirmed the desired compound had been made. It had mass spectrum: [MH⁺]=766.3 amu (calculated for C₃₈H₃₉N₉O₉ 765.78 amu).

5′-α-Chloroacetyl-2′(3′)-(2-TAMRA-amidoethylcarbamoyl)adenosine (13)

5′-Hydroxy-2′(3′)-(2-TAMRA-amidoethylcarbamoyl)adenosine 12 (2.0 mg, 2.6 μmol) was dissolved in DMF (250 μl) before chloroacetyl chloride (3 μl, 38 μmol) was added. The resulting red solution was stirred at room temperature for 1 hour. The desired compound 13 (1.4 mg, 1.7 μmol, 65%) was isolated by preparative HPLC. It had mass spectrum: [MH⁺]=842.3 amu (calculated for C₄₀H₄₀ClN₉O₁₀ 842.26 amu).

### Example 8

### 5′-FSB-acetyl-2′(3′)-(2-TAMRA-amidoethylcarbamoyl)adenosine (14)

5′-Hydroxy-2′(3′)-(2-TAMRA-amidoethylcarbamoyl)adenosine 12 (2.0 mg, 2.6 μmol) was dissolved in DMF (250 μl) before fluorosulfonylbenzoyl chloride hydrochloride (15 mg, 30.6 μmol) was added in 3 portions (over 45 minutes). Each addition of the flurosulfonylbenzoyl chloride was followed by addition of 5 μl of triethylamine. LC-MS indicated that after 1 hour, most of the starting material was consumed and preparative HPLC yielded 14. An analytical sample was submitted for HRMS and showed: [MH⁺]=952.2695 amu with an error of ±3.7 ppm (calculated for C₄₅H₄₂FN₉O₁₂S: 952.273 amu).

### Example 9

### EGF Receptor Tyrosine Kinase Labeling in A431 Cells

Plasma membranes containing the epidermal growth factor receptor (EGFR) were prepared from the A431 human epidermoid cancer cell line. Approximately 1×10⁸ A431 cells were resuspended in 10 mls 20 mM HEPES, pH 7.5, 0.14M NaCl, 5 mM KCl, 0.1 mM EDTA, 0.1% glucose, and 0.035% sodium bicarbonate. The cells were lysed by Dounce homogenization, and the lysate was diluted into 80 mls of 20 mM sodium borate, pH 10.2, 0.1 mM EDTA. This mixture was placed on a rocker at 4° C. for 10 minutes, and the membranes from the mixture were isolated by centrifugation at 16,000 rpm for 30 minutes in a Beckman JA17 rotor. The plasma membranes were further purified by centrifugation over a 40%/30% sucrose cushion at 30000 rpm for 60 minutes in a Beckman MLS 50 swinging bucket rotor. The plasma membranes were collected at the aqueous—sucrose interface, and solubilized in buffer containing 20 mM HEPES, pH 7.5, 50 mM NaCl, 20 mM MgCl2, 0.05 mg/ml BSA, and 0.1% Triton-X-100.

Solubilized A431 plasma membranes (˜0.5 mg total protein) were incubated with (5)(20 μM) for 3 hours. The sample was denatured by addition of urea to 8M, reduced by heating in the presence of DTT (10 mM) at 65° C. for 10 minutes, and then alkylated by incubating with iodoacetamide (50 mM), at 37° C. for 45 minutes. The alkylated sample was run over a BioRad DG column pre-equilibrated with 2M urea and 20 mM TrisCl pH 8.0, so as to dilute out the urea. SDS was added to the sample to a final concentration of 1%, and the sample was heated at 65° C. for 5 minutes. The sample was diluted with one volume 2×Binding buffer (2% Triton-X-100, 1% Tergitol, 300 mM NaCl, 2 mM EDTA, and 20 mM TrisCl; pH 7.4), The AX0231-labeled proteins were antibody captured by incubating the sample with 50 μl of anti-TAMRA monoclonal antibody beads overnight. The beads were washed three times with 1×Binding buffer, 0.2% SDS, and washed twice with 50 mM TrisCl, pH 8.0, 100 mM NaCl. The antibody captured proteins were eluted from the beads by adding one bed volume of non-reducing gel sample buffer to the beads, and heating the beads at 65° C. for 10 minutes. The captured proteins were resolved by SDS-PAGE electrophoresis. The gel was stained with Coomassie, and the band correponding to EGFR was cut from the gel.

The proteins in this band were trypsinized, and the tryptic peptides were extracted. The probe labeled tryptic fragments were captured with anti-TAMRA monoclonal antibody beads. After elution from the beads the captured fragment was analyzed by mass spectroscopy (MS) to identify the peptide sequence:

I P V A I K* E L R or

V K I P V A I K* E L R

* denotes site of labeling

Two sequences were identified as a result of the KVK sequence at the beginning of the fragment, trypsin cleavage generating two peptides. This sequence corresponds to amino acids 740-748 or 738-748 respectively in human EGFR kinase and is located within the active site of the protein.

### Example 10

### Assaying the Effect of Inhibitors on EGF Receptor Tyrosine Kinase Labeling in A431 Cells

Plasma membranes prepared as described above in Example 15 were treated with the ANBP (5), either alone (control), in the presence of an EGF receptor tyrosine kinase inhibitor (AG1478), or inhibitors that target other kinases (AG825 & AG 925), or ATP, and processed as described. Solubilized plasma membranes (˜0.5 mg/ml total protein) were then either treated with AX0231 alone (20 μM), or first pretreated with either AG1478 (5 μM), AG825 (50 μM), AG957 (50 EM), or ATP (3 mM), for 5 minutes, after which AX0231 was added (20 μM). After 60 minutes, the reaction was quenched by the addition of gel sample buffer. The samples were electrophoresed on a 10% SDS-PAGE gel. The gel was then scanned on a FMBIO2 gel scanner, and the intensity of labeling of the EGFR was quantitated by ImageAnalysis. Treatment with 5 μM of the EGF inhibitor reduced probe labeling by 90% while treatment with 50 μM of the other inhibitors produced significantly less inhibition.

### Example 11

### Labeling of Insulin Receptor Tyrosine Kinase

GST-fusion protein of the insulin receptor kinase domain (GST-InsRK) (50 ng), in the presence of boiled rat liver lysate (0.25 mg/ml), was either reacted with (5) (25 μM) alone for 30 minutes, or first pretreated with 25 or 250 μM FSBA (fluorosulfonylbenzoic acid) for 30 minutes, and then reacted with (5) (25 μM) for 30 minutes. The reaction was quenched by the addition of gel sample buffer. The samples were electrophoresed on a 10% SDS-PAGE gel. The gel was then scanned on a FMBIO2 gel scanner, and the intensity of labeling of the GST-InsRK was quantitated by ImageAnalysis. The results indicated that GST-InsRK was labeled by (5), which labeling was inhibited by FSBA.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

We claim:

1. A method for determining a profile of one or more target adenosine nucleotide-binding proteins in a complex protein mixture, employing probes comprising adenosine conjugated with a fluorescent tag (“TAG”) through a linker of at least two atoms to oxygen at one position of ribose and a reactive group that reacts with an amino acid functionality at a second position when said probe is bound to said target adenine nucleotide-binding protein(s), said method comprising:
combining in a reaction medium said probe and said complex protein mixture under conditions of reaction of said probe with said target adenine nucleotide-binding protein(s), whereby a conjugate of said probes and said target adenine nucleotide-binding protein(s) is (are) formed; and
determining said adenine nucleotide-binding protein profile by sequestering said conjugate or detecting fluorescence from said TAG.

2. A method according to claim 1, wherein said reactive group is selected from the group consisting of fluorosulfonyl, vinylsulfonyl, acryl, and chloroacetyl.

3. A method according to claim 1, wherein said TAG is linked to said adenosine at the 2′ or 3′ position or mixture thereof.

4. A method according to claim 1, wherein said TAG is linked to said adenosine through an amidoalkylester group.

5. A method according to claim 1, wherein said probe binds to a plurality of target adenine nucleotide-binding proteins.

6. A method for determining a profile of one or more target adenosine nucleotide-binding proteins in a complex protein mixture, employing probes comprising adenosine conjugated with a fluorescent TAG through a linker of at least two atoms to oxygen at one position of ribose and a reactive group that reacts with an amino acid functionality at a second position when said probe is bound to said adenine nucleotide-binding protein(s), said probes of the formula: 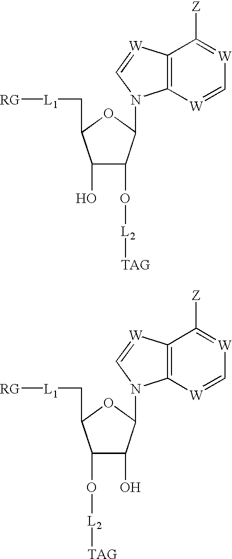
wherein:
each W is independently carbon or nitrogen;
Z is hydrogen or amino;
RG is a reactive group capable of reacting with at least one of thiol, hydroxyl, carboxyl or amino joined through L₁ to the 5′ carbon of the ribose, where the reactive group isselected from the group consisting of fluorosulfonyl, fluorophosphonyl ester, halogen, epoxide, ethylene α to an activating group, and halogen β to an activating group; and
L₁ and L₂ are optionally present and are independently alkyl or heteroalkyl groups of 1-20 backbone atoms selected from the group consisting of —N(R)—, —O—, —S— or —C(R)(R)—, where each R is independently H or —C₁₋₆ alkyl straight or branched chain; and
TAG is a detectable label joined to the oxygen of the 2′ or 3′ position of the ribose through linking group L₂;
said method comprising:
combining in a reaction medium said probe and said complex protein mixture under conditions of reaction of said probe with said target adenine nucleotide-binding protein(s), whereby a conjugate of said probes and said target adenine nucleotide-binding protein(s) is formed; and
determining said adenine nucleotide-binding protein profile by sequestering said conjugate or detecting fluorescence from said TAG.

7. A method according to claim 6, wherein said reactive group is selected from the group consisting of fluorosulfonyl, vinylsulfonyl, acryl, and chloroacetyl.

8. A method according to claim 6, wherein said TAG is linked to said adenosine at the 2′ or 3′ position or mixture thereof.

9. A method according to claim 6, wherein said TAG is linked to said adenosine through an amidoalkylester group.

10. A method according to claim 6, wherein said probe binds to a plurality of target adenine nucleotide-binding proteins.

11. A method according to claim 6, wherein said probe is 5′-fluorosulfonylbenzoylamido-2′(3′)-(2-TAMRA-amidoethylcarbamoyl)adenosine.

12. A compound of the formula: 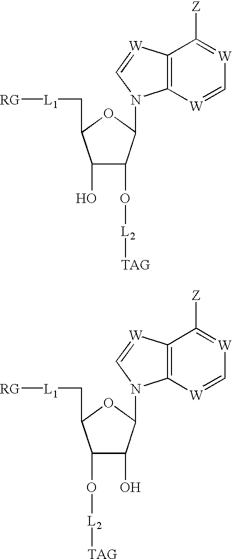
wherein:
each W is independently carbon or nitrogen;
Z is hydrogen or amino;
RG is a reactive group capable of reacting with at least one of thiol, hydroxyl, carboxyl or amino joined through L to the 5′ carbon of the ribose, where the reactive group is selected from the group consisting of fluorosulfonyl, fluorophosphonyl ester, halogen, epoxide, ethylene α to an activating group, and halogen β to an activating group;
L₁ and L₂ are optionally present and are independently alkyl or heteroalkyl groups of 1-20 backbone atoms selected from the group consisting of —N(R)—, —O—, —S— or —C(R)(R)—, where each R is independently H or —C₁₋₆ alkyl straight or branched chain; and
TAG is a detectable label joined to the oxygen of the 2′ or 3′ position of the ribose through linking group L₂.

13. A compound according to claim 12, wherein said TAG is a fluorescer.

14. A compound according to claim 12, wherein said TAG is a ligand.

15. A compound according to claim 12, wherein said L₁ or L₂ is aliphatic.

16. A compound according to claim 12 of the formula 5′-fluorosulfonylbenzoylamido-2′(3′)-(2-TAMRA-amidoethylcarbamoyl)adenosine.

17. A conjugate of a compound according to claim 16 with a target adenosine nucleotide-binding protein.

18. A conjugate of a compound according to claim 12 with a target adenosine nucleotide-binding protein.

19. A method for determining a profile of one or more adenine nucleotide-binding proteins in a complex protein mixture, comprising:
detecting a signal from one or more probe-target adenine nucleotide-binding protein conjugates formed by contacting said complex protein mixture with one or more probes comprising adenosine conjugated (i) via a linker to a detectable label and (ii) to a reactive group that reacts with an amino acid in a corresponding target adenine nucleotide-binding protein when said probe is bound to said adenine nucleotide-binding protein, to form said one or more probe-target conjugates.

20. A method for determining a profile of one or more target adenosine nucleotide-binding proteins in a complex protein mixture, comprising:
sequestering one or more probe-target adenosine nucleotide-binding protein conjugates formed by contacting said complex protein mixture with one or more probes comprising adenosine conjugated (i) via a linker to a TAG and (ii) to a reactive group that reacts with an amino acid in a corresponding target adenine nucleotide-binding protein when said probe is bound to said adenine nucleotide-binding protein, to form said one or more probe-target adenosine nucleotide-binding protein conjugates, by binding said TAG to a cognate receptor.

21. A method according to claim 19 or 20, wherein said reactive group is selected from the group consisting of fluorosulfonyl, vinylsulfonyl, acryl, and chloroacetyl.

22. A method according to claim 19 or 20, wherein said TAG is linked to said adenosine at the 2′ or 3′ position or mixture thereof.

23. A method according to claim 19 or 20, wherein said TAG is linked to said adenosine through an amidoalkylester group.

24. A method according to claim 19 or 20, wherein said probe binds to a plurality of target adenine nucleotide-binding proteins.

25. A method according to claim 19 or 20, wherein said probe is of the formula: 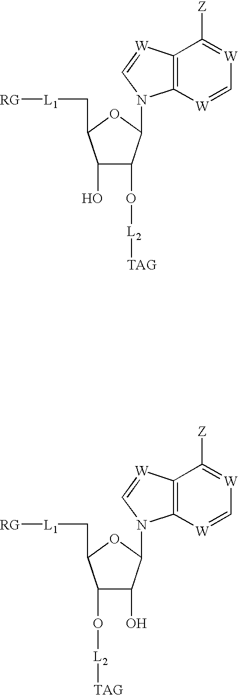
wherein:
each W is independently carbon or nitrogen;
Z is hydrogen or amino;
RG is reactive group capable of reacting with at least one of thiol, hydroxyl, carboxyl or amino joined through L to the 5′ carbon of the ribose, where the functional group may be directly bonded to L or through a link, the reactive group being a single functional group or a combination of functional groups comprising, halogen, O, S, N, P, and C, selected from the group consisting of fluorosulfonyl, fluorophosphonyl ester, halogen, epoxide, ethylene α to an activating group, and halogen β to an activating group;
L₁ and L₂ are optionally present and are independently alkyl or heteroalkyl groups of 1-20 backbone atoms selected from the group consisting of —N(R)—, —O—, —S— or —C(R)(R)—, where each R is independently H or —C₁₋₆ alkyl straight or branched chain; and
TAG is a detectable label joined to the oxygen of the 2′ or 3′ position of the ribose through linking group L₂.

26. A method according to claim 25, wherein said probe is 5′-fluorosulfonylbenzoylamido-2′(3′)-(2-TAMRA-amidoethylcarbamoyl)adenosine.
